(19) United States
(12) Patent Application Publication
(10) Pub. No.: US 2017/0027481 A1
(43) Pub. Date: Feb. 2, 2017

(54) ORGANIC ELECTROCHEMICAL SENSOR FOR MEASURING BODY PARAMETERS

(71) Applicant: HFT SMARTSENSORS, INC., ROCKFORD, IL (US)

(72) Inventors: NICOLA COPPEDE', ROCKFORD, IL (US); ANDREA ZAPPETTINI, ROCKFORD, IL (US); LAURA MARCHINI, ROCKFORD, IL (US)

(21) Appl. No.: 15/223,071

(22) Filed: Jul. 29, 2016

(30) Foreign Application Priority Data
Jul. 29, 2015 (IT) 102015000039416

(51) Int. Cl. A61B 5/1486; A61B 5/00; A61B 5/145

(52) U.S. Cl. CPC A61B 5/1486; A61B 5/14532; A61B 5/14546; A61B 5/6804; A61B 5/681; A61B 5/742; A61B 2562/125; A61B 2560/0475

(57) ABSTRACT

The present invention relates to an OECT sensor (1) for determining biochemical parameters in a subject's perspiration, comprising a filament (2) coated with a first layer (3) made of a conductive polymer, wherein the ends of the filament (2) are connected to two electrodes (4, 4′), wherein a first electrode (4) is grounded (V₀), while a negative potential (V−) is applied to the second electrode (4′), the sensor (1) further comprising a control electrode (5), to which a positive potential (V+) is applied, wherein the filament (2) comprises a second layer (6) comprising an enzyme which catalyzes a transformation reaction of an analyte present in the liquid to be analyzed with generation of cations.

The present invention relates to an organic electrochemical transistor sensor, generally known as OECT, for measuring body parameters, obtained on textile fabric comprising an appropriately integrated enzyme adapted to catalyze the reaction of an analyte with generation of a cation species.

### BACKGROUND ART

Monitoring body parameters, in particular during physical activity, is current practice for health and for sports reasons.

Parameters such a blood pressure or heart rate, which provide an indication of the subject's physical state, in particular as a function of the physical effort which is being sustained, are normally monitored. Such a monitoring is performed by applying electrodes to the subject's body connected by means of an appropriate wiring to a storage and processing system of the collected data.

More recently, in order to solve the problem of dimensions of such devices, miniaturized sensors have been suggested, connected to a data receiver in wireless mode.

However, monitoring the blood pressure and heart rate parameters has been deemed insufficient to evaluate the global physical state of a subject, in particular of a healthy subject performing sports activities. Indeed, in such a case, it would be useful to monitor some biochemical parameters, indicative of the energy consumption, the muscular fatigue and the dehydration level to which the athlete is subjected.

In order to solve this problem sensors based on an electrochemical organic transistor device have been suggested, consisting of a cotton fiber coated with a conductive polymer, which maintains the features of total wearability and can directly absorb the fluid to be analyzed. The two ends of the fiber are connected with two “source” and “drain” electrodes, and a control electrode (“gate”) consisting of a metal wire, e.g. made of silver or platinum (G. Tarabella et al., J. Mater. Chem., 2012, 22, 23830-23834; N. Coppedè et al., J. Mater, Chem. B, 2014, 2, 5620-5626). Thereby, a biochemical parameter can be determined by putting a drop of body fluid, e.g. perspiration, in contact with the conductive fiber and the control electrode.

This type of sensor, however, allows to determine only some major parameters, in particular the saline concentration in the body fluid or the concentration of biological molecules, such as adrenalin, melanin or dopamine.

The need exists to design a sensor which can also selectively determine parameters closely related to physical effort, such as energy expenditure or muscular fatigue which could be integrated in garments, so as to monitor said biochemical parameters in a subject in a continuous, efficient and non-invasive manner.

### SUMMARY OF THE INVENTION

It is thus an object of the present invention a device for measuring the biochemical parameters of a subject, comprising an organic electrochemical sensor, which comprises a textile fiber filament coated with a conductive polymer, the source and drain electrodes connected to said filament and a gate control electrode, and wherein said filament comprises an enzyme, appropriately fixed onto the polymer, capable of catalyzing the reaction or an analyte not selectively detectable by said sensor per se, said reaction generating a cation species which is conversely detectable by the organic electrochemical sensor.

It is another object a garment which comprises the device for measuring biochemical parameters of a subject comprising an organic electromechanical sensor as defined above.

It is a further object of the invention a method for manufacturing the sensor of the invention.

The present invention thus relates to an organic electrochemical sensor and a garment as set out in the accompanying claims, the definitions of which form an integral part of the present description.

Further features and advantages of the present invention will become apparent from the description of a preferred embodiment, given here by way of non-limiting example, with reference to the following drawings, in which:

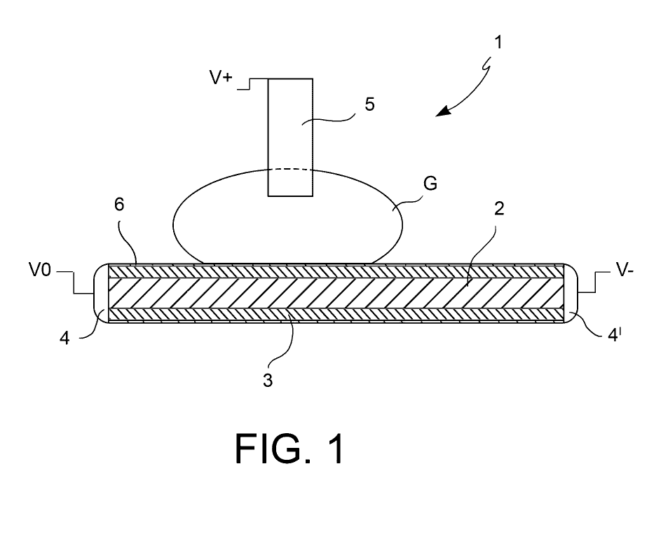

FIG. 1 is a diagrammatic section view of the sensor which is the object of the invention;

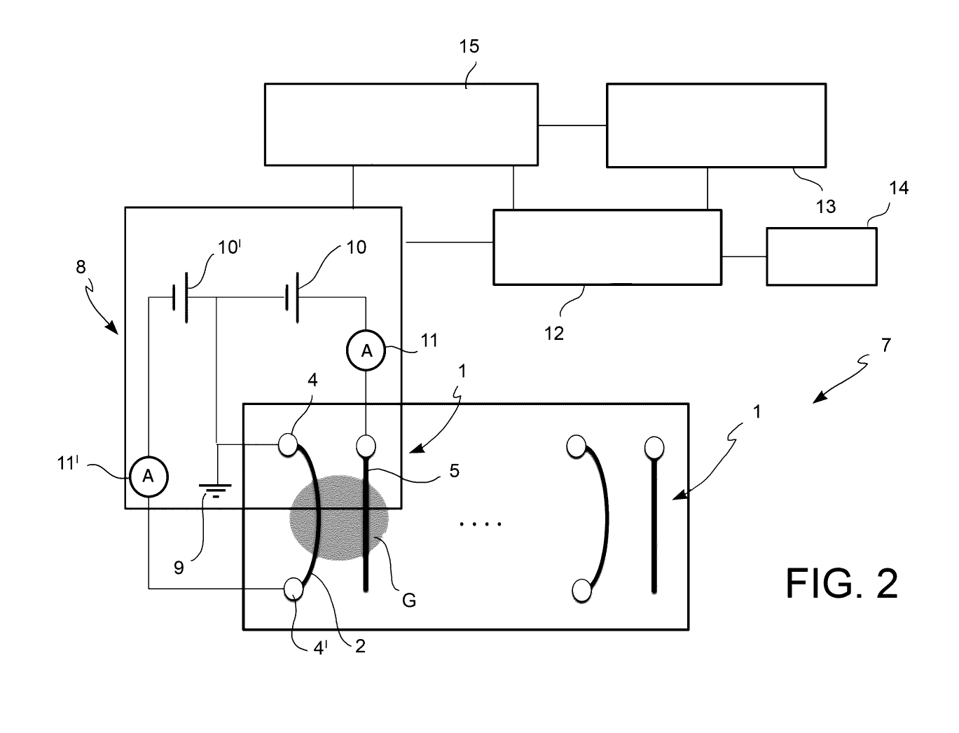

FIG. 2 is a diagrammatic view of the device of the invention comprising the sensor which is the object of the invention;

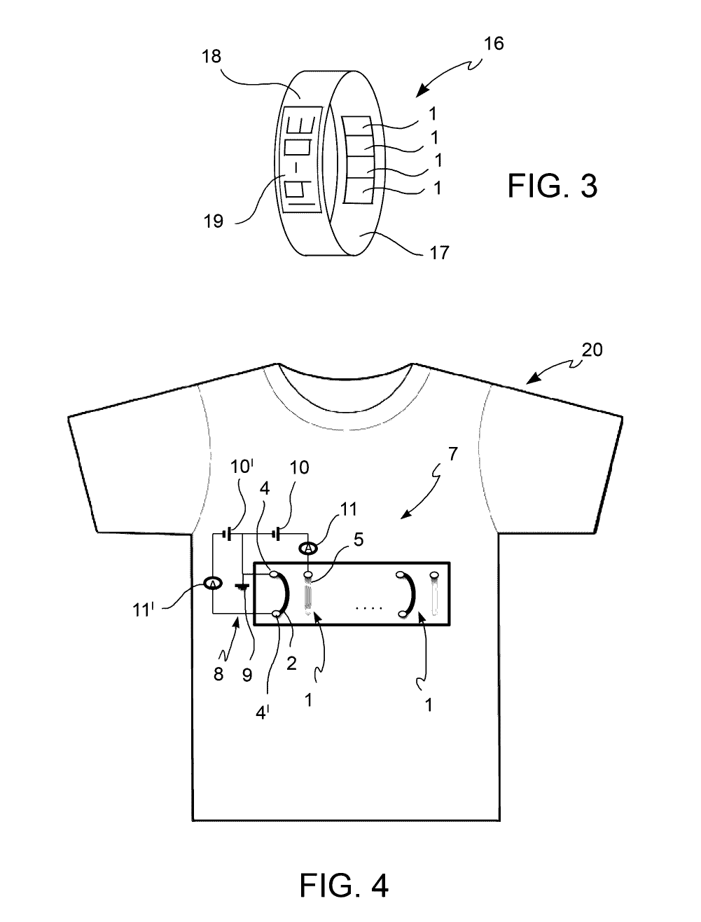

FIG. 3 is a perspective view of a first example of garment which comprises the device of the invention.;

FIG. 4 is a diagrammatic view of a second example of garment comprising the device of the invention.

### DETAILED DESCRIPTION OF THE INVENTION

With reference to FIG. 1, the sensor according to the invention, indicated by reference numeral 1 as a whole, comprises a filament 7 of a textile fiber coated with a first layer 3 made of a conductive polymer and a second layer 6 comprising an enzyme which catalyzes a transformation reaction of an analyte present in the liquid to be analyzed with generation of cations.

The ends of filament 2, the length of which may be fixed as desired, but being generally of about 1 cm, are connected to two electrodes 4, 4′, wherein a first electrode 4 is grounded (V₀), while a negative potential (V−) is applied to the second electrode (4′), preferably from −0.1 Volt to −0.01 Volt.

Sensor 1 further comprises a control electrode 5, to which a positive potential (V+) is applied, preferably from 0.2 to 1 Volt. The control electrode 5 is positioned with respect to filament 2 so that the two ends of the filament form the “drain” and “source” of a transistor, while the control electrode 5 forms the “gate” of the transistor when a drop G of the liquid to be analyzed is simultaneously put into contact with the filament 2 and the control electrode 5 (see FIG. 1).

In some embodiments, filament 2 consists of a natural textile fiber selected from cotton, silk, wool and flax or a synthetic fiber, such as nylon or acrylic material.

In some embodiments, the conductive polymer which forms layer 3 is selected from polymers based on poly(3,4-ethylenedioxythiophene) (PEDOT), poly(6-(thiophen-3-yl)hexan-1-sulfonate (PTHS), polvaniline, polypyrrole, polythiophene and polyfuran. Preferably, the conductive polymer is selected from. PEDOT:PSS (poly(3,4-ethylendioxythiophen)-polystyrene sulfonate) and PEDOT:TOS (poly(3,4-ethylenedioxythiophen)tosylate).

In preferred embodiments, layer 3 is obtained by:

a) an aqueous solution of PEDOT:PSS containing from 2% to 30%, preferably about 21%, by volume of ethylene glycol and 1% by volume of dodecylbenzenesulfonjc acid as a surfactant. The presence of a surfactant promotes the adhesion of the polymer to the silk filament, or

b) an aqueous solution of PEDOT:TOS containing from 2% to 30%, preferably about 21%, by volume of ethylene glycol and 1% by volume of dodecylbenzenesulfonic acid, or

c) an aqueous solution of 1% by weight PTHS containing 6% by volume of ethylene glycol and 1% by volume of 3-(glycidyl propyl)trimethoxysilane (GOPS),

The coating of the filament with the conductive polymer may be performed by dipping the filament, either alone integrated in a textile material, into a conductive polymer solution as set out above and then drying the coated filament thus obtained for about 1 hour at 120-150° C.

The layer 3 made of conductive polymer preferably has a thickness from 50 to 200 nm. The filament thus coated has an electric conductivity from 80 to about 400 Ohm/cm.

The layer 6 containing the enzyme may be obtained according to one of the following methods:

i) dipping filament 2 coated with layer 3, either alone or Integrated in a textile material, into a solution of the enzyme and subsequent drying at room temperature for approximately 2 hours, or

ii) dipping filament 2 coated with layer 3, either alone or integrated into a textile material, into a solution of enzyme and chitosan for about 5 hours, and subsequent drying at room temperature, or

ii) dipping filament 2 coated with layer 3, either alone or integrated into a textile material, firstly into a solution of chitosan as defined below and then into a solution of enzyme, as defined above, followed by drying at room temperature, or

iv) dipping filament 2 coated with layer 3, either alone or integrated in a textile material, into solution of polyglycidyl methacrylate (PGMA) and poly(2-hydroxyethylmethacrylate) (PHEMA) mixed with enzyme.

The solution of chitosan has a concentration of about 5 mg/ml and may be prepared, for example, by dissolving chitosan (0.5 g) in an aqueous solution of acetic acid (100 ml, 5 mM, ph=5-6). 5 μL of a solution of enzyme (concentration 8 mg/ml) are added to 50 μ1 of solution of chitosano, and the resulting solution is buffered with PBS solution (Phosphate Buffered Saline, pH=7.2).

The employed enzyme may be of various type and will depend on the analyte it is intended to be analyzed. In some embodiments, the enzyme is selected from: glucose oxidase (GOx) for determining glucose, lactate oxidase (LOx) for determining lactic acid, and urease for determining urea.

In preferred embodiments, the layer 3 made of conductive polymer further contains a silane compound having the function of fixing the enzyme to the conductive polymer.

More preferably, the silane compound is Trimethoxy[2-(7-oxabicyclo[4.1.0]hept-3-yl)ethyl]silane having formula

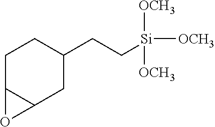 [SMILES: CO[Si](CCC1CCC2OC2C1)(OC)OC]

The silane molecule allows t reduce the PEDOT swelling, by forming a network of bonds which tend to fix the silane, especially in the presence of amines which are found on the thread. Thereby, the silane molecule tends to fix the polymer to the thread in a stable and definitive manner. The methoxyl CH₃O— groups, bound to the atom of Si, are the reactive part of the silane and are subjected to hydrolysis thus forming more reactive species. The latter may be bound to the conductive polymer and/or to another silane in a polymerization process. The epoxide instead may be used for the conjugation reaction of the enzymatic proteins, thus promoting the formation of stable bonds which ensure the permanence of the enzyme on the surface of layer 3 made of silane and conductive polymer, e.g. PEDOT:PSS. Thereby, by wetting the thread with the enzyme in the presence of silane, the enzyme can be fixed to the surface of the conductive polymer in a definitive manner.

In this embodiment, layer 3 is thus made according to the methods described above, using any one of the previously outlined solutions a), b) or c), but in which an amount from 1% to 5% by weight of a silane compound as set out above was added.

The control electrode 5 consists of a noble metal or of a textile fiber filament coated with a conductive polymer as set out above. Preferably, the control electrode 5 is a platinum wire.

The basic operating principles of the organic electrochemical sensor are described in. G. Tarabella at al., J. Mater. Chem., 2012, 22, 23830-23834 and in N. Coppedè at al., J. Mater. Chem. 13, 2014, 2, 5620-5626, in relation to two different embodiments of the sensor in different applications.

Without being bond to a particular theory, the operation of a sensor 1 comprising GOx or LOx includes the oxidation reaction to layer 6 (containing the enzyme)—liquid interface, respectively, of D-glucose to give D-glucose-1,5-lactone or L-lactate to give pyruvate, in both cases with formation of H₂O₂. The H₂O₂ then oxidizes at the control electrode 5—liquid interface to give oxygen and protons which are then conveyed, by the potential V+ of the control electrode 5, towards the conductive polymer, subjected to a difference of potential V_{drain/source}=V₋−V₀, and de-dope the polymer according to the following reaction:

Pol⁺:X⁻+M⁺+e⁻ [symbol] Pol+M⁺:X⁻

where Pol⁺:X⁻ is one of the conductive polymers listed above, M⁺ is a cation present in the drop G of liquid to be analyzed, in the specific case a proton, and e⁻ is an electron.

The consequence of de-doping the conductive polymer is a decrease of the current intensity I_{drain/source} along filament 2. The concentration of the cation M⁺ in the liquid to be analyzed, and thus of the initial analyte, may thus be determined by means of appropriate calibration curves of the sensor, in which the variation of the I_{drain/source} is put into relation with a series of predetermined concentrations of the analyte in the sample. Thereby, the concentration of the analyte, in the examples shown glucose or lactate, can be determined in the drop G of fluid to be analyzed.

The reaction of D-glucose catalyzed by GOx and of lactic acid catalyzed by LOx may be further accelerated by adding ferrocene, which has the function of co-catalyst, to the enzyme solution used in the preparation of layer 6. In this case, the mechanism includes to de-dope the conductive polymer by the electrons lost by the D-glucose or L-lactate in the oxidation reaction.

The de-doping reaction is reversible, so that the ions scatter from Pol⁺:X⁻ towards the solution reforming the initial conductive polymer, which causes an increase of the when the positive potential V+ is no longer imposed on the control electrode 5.

FIG. 2 shows a device 7 in which one or more sensors 1 are integrated. Sensors 1 may be of the same type, i.e. adapted to determine the same analyte in the various points of the body in which they are applied, or preferably be of different type, so as to analyze multiple biochemical parameters of a subject, such as for example concentration of D-glucose, lactic acid and urea.

Each sensor 1 is operatively connected to a measuring circuit 8 which comprises:

a ground connection 9 connected to the first electrode 4 at one end of filament 2,

a first voltage generator 10, adapted to generate a positive voltage on the control electrode 5,

a second voltage generator 10′, adapted to generate a negative voltage on the second electrode 4′ connected to the other end of filament 2,

a first ammeter 11, adapted to measure the current intensity in the circuit connected to the control electrode 5,

a second ammeter 11′, adapted to measure the current intensity in the circuit connected to filament 2.

The first and second current generators 10, 10′ are preferably a thin lithium battery.

The measuring circuit 8 is operatively connected to a memory 12 for data recording, which in turn is connected to a data transmission circuit 13 and, optionally, to a display 14.

Memory 12, circuit 13 and display 14 are also connected to a voltage generator 14, preferably a thin lithium battery.

The device 7 diagrammatically shown herein may be implemented in a garment to be applied to the body of a subject for measuring the aforesaid biochemical parameters of the latter.

FIG. 3 shows a first embodiment of such a garment, i.e. a wrist band 16.

The wrist band. 16 is typically an elastic band capable of adhering to the wrist of a subject. The elastic band may be made of elastic, either synthetic or natural fabric, or of an elastomer. At least one inner face 17 of the wrist band 16 comprises one or more sensors 1, as defined above, while the circuits 8 and 13, the memory 12 and the battery 15 are preferably embedded in the body of the wrist band 16.

Display 19, which allows the user to monitor the measured biochemical parameters directly, is arranged instead on the outer face 18 of the wrist band 16.

In some embodiments, sensors 1 may be removable. For example, sensors 1 may be associated with the inner face 17 of the wrist band 16 by means of tear or clip systems.

FIG. 5 shows a second embodiment of a garment according to the invention, i.e. a T-shirt 20.

T-shirt 20 is made of elastic material, typically an elastic fabric made of natural or synthetic material, so as to adhere to the body. Figure shows a T-shirt, but there is nothing to prevent such a garment from being an. A-shirt or a long-sleeved sweater or simply a chest strap.

One or more sensors 1 as previously described are arranged on the inner side of the T-shirt 20 (i.e. on the side in contact with the body of the subject who is wearing it). The sensors are operatively connected to circuit 8, which in turn is connected to the circuits 12 and 13 and to battery 15 (not shown). In this case, display 14 may be omitted.

The sensors may be positioned on the front, back or side. In particular, the sensors 1 are arranged at the points with the highest perspiration or where perspiration collects the most, so as to promote the reading of the desired biochemical parameters.

All the electric circuits and the battery will be preferably enclosed in a plastic material casing so as to isolate them from the perspiration. Furthermore, each casing is preferably associated with the garment in a removable manner, e.g. by means of appropriate clips, so as to remove it when the garment must be washed.

The invention allows to achieve the predetermined objects.

Device 7 may comprise a plurality of sensors so as to monitor various biochemical parameters at the same time.

For example, the sensor 1 for measuring the levels of :Lactic acid in the perspiration allows to evaluate the effort and the muscular fatigue of the person performing physical activity, e.g. an athlete.

The sensor for measuring glucose in the perspiration allows to evaluate the energy reserves of the body.

The fact that device 7 is miniaturized and integrated in a garment allows to monitor the subject's biochemical parameters without causing disturbance or annoyance to the subjects themselves, especially during physical and sports activities in general.

The sensor according to the invention is not.

exclusively dedicated to be used on a garment. Indeed, it may be used in all those application which require measuring an analyte in a fluid, in which the analyte may cause an enzymatic reaction with generation of cation species. The employed enzyme be any specific enzyme for a given analyte, the embodiments described above being only examples.

For example, the sensor 1 of the invention may be used for determining an analyte as described above in a biological fluid, such as blood plasma, urine and lacrimal secretions or secretions of various origin, by means of a device which typically cannot be applied to the body of the subject, but will be arranged in remote position.

Moreover, it will be possible to analyze non-biological fluids, such as for example tap or mineral water, waste water or other liquids from particular processes.

It is apparent that only some particular embodiments of the present invention have been described, to which those skilled in the art will be able to make all the changes required to adapt it to particular applications, without therefore departing from the scope of protection of the present invention.

1. An organic electrochemical sensor (1) for determining biochemical parameters in a liquid to be analyzed, comprising a filament (2) coated with a first layer (3) made of a conductive polymer, wherein the ends of the filament (2) are connected to two electrodes (4, 4′), wherein a first electrode (4) is grounded (V₀), while a negative potential (V−) is applied to the second electrode (4′), the sensor (1) further comprising a control electrode (5), to which a positive potential (V+) is applied, the control electrode (5) being positioned with respect to the filament (2) so that the two ends of the filament form the “drain” and “source” of a transistor, while the control electrode (5) forms the “gate” of the transistor when a drop (G) of the liquid to be analyzed is simultaneously put into contact with the filament (2) and the control electrode (5), characterized in that the filament (2) comprises a second layer (6) comprising an enzyme which catalyzes a transformation reaction of an analyte present in the liquid to be analyzed with generation of cations.

2. A sensor (1) according to claim 1, wherein said negative potential (V−) applied to the second electrode (4′) is from −0.1 Volt to −0.01 Volt and/or said positive potential (V+) applied to said control electrode (5) is from 0.2 to 1 Volt.

3. A sensor (1) according to claim 1, wherein the conductive polymer forming the layer (3) is selected from a polymer based on poly(3,4-ethylenedioxythiophene) (PEDOT), poly(6-(thiophen-3-yl)hexan-1-sulfonate (PTHS), polyaniline, polypyrrole, polythiophene and polyfuran, preferably from PEDOT:PSS (poly(3,4-ethylendioxythiophen)-polystyrene sulfonate) and PEDOT:TOS (poly(3,4-ethylenedioxythiophen)tosylate).

4. A sensor (1) according to claim 3, wherein the first layer (3) made of conductive polymer has a thickness from 50 to 200 nm and wherein the filament (2) coated with such a conductive polymer has an electrical conductivity from 80 to about 400 Ohm/cm.

5. A sensor (1) according to claim 1, wherein the first layer (3) made of conductive polymer comprises a silane compound.

6. A sensor (1) according to claim 5, wherein the silane compound is trimethoxy[2-(7-oxabicyclo[4.1.0]hept-3-yl)ethyl]silane.

7. A sensor (1) according to claim 1, wherein the control electrode (5) consists of a noble metal wire or by a textile fiber filament coated with a conductive polymer as set out in claim 3.

8. A sensor (1) according to claim 1, wherein said textile fiber is a natural textile fiber selected from cotton, silk, wool and flax or a synthetic fiber, such as nylon or acrylic material.

9. A sensor (1) according to claim 1 wherein the second layer (6) comprises a co-catalyst.

10. A sensor (1) according to claim 9, wherein the enzyme is selected from: glucose oxidase (GOx) for determining glucose, lactate oxidase (LOx) for determining lactic acid, and urease for determining urea; and wherein the co-catalyst is ferrocene both for GOx and for LOx.

11. A device (7) comprising one or more sensors (1) according to any one of claims from 1 to 10, wherein the sensors (1) can be of the same type, for determining the same analyte in various points of the body to which they are applied, or of a different type, for analyzing several biochemical parameters of a subject, wherein each sensor (1) is operatively connected to a measuring circuit (8) which comprises:
a ground connection (9) connected to the first electrode (4) at one end of the filament (2),
a first voltage generator (10), adapted to generate a positive voltage on the control electrode (5),
a second voltage generator (10′), adapted to generate a negative voltage on the second electrode (4′) connected to the other end of the filament (2),
a first ammeter (11), adapted to measure the current intensity in the circuit connected to the control electrode (5),
a second ammeter (11′), adapted to measure the current intensity in the circuit connected to the filament (2).

12. A device (7) according to claim 11, wherein the measuring circuit (8) is operatively connected to a memory (12) for data recording, which in turn is connected to a data transmission circuit (13) and, optionally, to a display (14).

13. A garment comprising a device (7) according to claim 11, said garment being selected from a wrist band (16), a T-shirt (20), an ankle band and a chest strap.

14. A process for manufacturing a sensor (1) as defined in claim 1, comprising the following steps:
1) dipping the filament (2), either alone or integrated into a textile material, into a conductive polymer solution as defined in claim 3, wherein the conductive polymer solution is preferably selected from:
a) an aqueous solution of PEDOT:PSS containing 21% by volume of ethylene glycol and 1% by volume of dodecylbenzenesulfonic acid as a surfactant, optionally containing the silane compound of claim 5 or 6 in an amount from 1% to 5% by weight,
b) an aqueous solution of PEDOT:TOS containing 21% by volume of ethylene glycol and 1% by volume of dodecylbenzenesulfonic acid, optionally containing the silane compound of claim 5 or 6 in an amount from 1% to 5% by weight,
c) an aqueous solution of 1% by weight PTHS containing 6% by volume of ethylene glycol and 1% by volume of 3-(glycidyl propyl)trimethoxysilane (GOPS), optionally containing the silane compound of claim 5 or 6 in an amount from 1% to 5% by weight;
2) drying the coated filament (2) thus obtained for about 1 hour at 120-150° C.,
3) dipping the filament (2) coated with layer (3), either alone or integrated in a textile material, into a solution of enzyme and chitosan for about 5 hours, or dipping the filament (2) coated with the layer (3), either alone or integrated in a textile material, firstly into a solution of chitosan and then into a solution of enzyme, or dipping the filament (2) coated with the layer (3), either alone or integrated in a textile material, into a solution of polyglycidyl methacrylate (PGMA) and poly(2-hydroxyethylmethacrylate) (PHEMA) mixed with enzyme,
4) drying the filament (2) coated with said first layer (3) and with said second layer (6) at room temperature.
